(19) United States
(12) Patent Application Publication
(10) Pub. No.: US 2022/0002333 A1
(43) Pub. Date: Jan. 6, 2022

(54) CRYSTALLINE FORM A OF 2'-FLUORO-4'-SUBSTITUTED NUCLEOSIDE ANALOGUE I AND PREPARATION METHOD AND APPLICATION THEREOF

(71) Applicant: Henan Genuine Biotech Co., Ltd., Pingdingshan (CN)

(72) Inventors: Junbiao CHANG, Zhengzhou (CN); Jinfa DU, Zhengzhou (CN); Dongxu YI, Zhengzhou (CN)

(21) Appl. No.: 17/475,377

(22) Filed: Sep. 15, 2021

Related U.S. Application Data
(63) Continuation-in-part of application No. PCT/CN2019/083217, filed on Apr. 18, 2019.

(30) Foreign Application Priority Data
Mar. 15, 2019 (CN) 201910197742.0

(51) Int. Cl. C07H 19/06; A61P 31/12; A61P 35/00

(52) U.S. Cl. CPC C07H 19/06; C07B 2200/13; A61P 35/00; A61P 31/12

(57) ABSTRACT

A crystalline form A of a compound I is a monoclinic system and a space group thereof is p2₁.

### CROSS-REFERENCE TO RELATED APPLICATIONS

This application is a continuation-in-part of International Patent Application No. PCT/CN2019/083217 with an international filing date of Apr. 18, 2019, designating the United States, now pending, and further claims foreign priority benefits to Chinese Patent Application No. 201910197742.0 filed Mar. 15, 2019. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, Mass. 02142.

### BACKGROUND

The disclosure relates to 2′-fluoro-4′-substituted nucleoside analogue I, and more particularly to a crystalline form A of 2′-fluoro-4′-substituted nucleoside analogue I, and preparation method and application thereof, which belongs to the field of pharmaceutical chemistry.

Some conventional nucleoside analogues exhibit significant antiviral activity, for example, to fight against human immunodeficiency virus (HIV), hepatitis B virus (HBV) and hepatitis C virus (HCV), and thus are clinically used for the treatment of viral infections. In addition to the antiviral activity, some nucleosides also have anticancer activity. However, these drugs have certain defects. On the one hand, the efficacy of the drugs is limited, on the other hand, long-term use thereof poses serious toxicity and side effects and will produce drug resistance. Therefore, the design and synthesis of new nucleoside analogues is an important research direction for the discovery of new antiviral drugs.

Studies show that 2′-fluoro-4′-substituted nucleoside analogues have antiviral activity, and the clinical dosage for adults is only 1-3 mg. It is well-known that the solubility and bioavailability of a drug are subject to the crystalline forms thereof. In addition, the stability, fluidity and compressibility thereof may also be different for different crystalline forms. These physical and chemical properties have a certain impact on the application of the drug, thus affecting the therapeutic effect of the drug. To ensure the quality of tablets and clinical medication effect, a crystalline form of a drug with stable performance and easy to mix with excipients should be developed. Therefore, studying the crystalline form of 2′-fluoro-4′-substituted nucleoside analogue I is conducive to its use in drug processing and pharmaceutical composition.

### SUMMARY

One objective of the disclosure is to provide a crystalline form A of 2′-fluoro-4′-substituted nucleoside analogue I. Another objective of the disclosure is to provide a preparation method of the crystalline form A and an application of the crystalline form A for preparation of antiviral drugs, particularly for anti-HIV drugs. Still another objective of the disclosure is to provide an application of the crystalline form A in the preparation of antitumor drugs, particularly in the preparation of drugs fighting against lung cancer, gastric cancer, colorectal cancer or lymphatic cancer, non-Hodgkin's lymphoma and leukemia.

2′-fluoro-4′-substituted nucleoside analogue I of the disclosure is represented by the following formula:

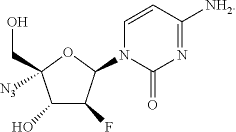 [SMILES: [N-]=[N+]=N[C@]1(CO)O[C@@H](n2ccc(N)nc2=O)[C@@H](F)[C@@H]1O]

The crystalline forms of the compound were systematically screened by means of unitary method, binary method, cooling, dissolution (positive and negative), diffusion and so on, and two crystalline forms were found: crystalline form A and amorphous crystalline form B. The stabilities of the crystalline form A and the crystalline form B under high temperature, high humidity and light were evaluated respectively. The evaluation results showed that crystalline form B could be gradually transformed into crystalline form A within ten days under high humidity and high temperature conditions, and the crystalline form A was stable under the same conditions. These experimental conditions were as follows: the high temperature condition is 60° C. for five days and ten days; the high humidity condition is 40° C. and 75% humidity for five and ten days; the light conditions were 4500 lux for five and ten days.

The compound I is dissolved in methanol, ethanol, isopropanol, ethyl acetate, dichloromethane or other solvents, and then the solvent is removed by a rotary evaporator to obtain a sheet amorphous solid B. The sheet amorphous solid B has obvious static electricity and is easily adsorbed on the container wall. After 10 days of storage at 40° C./75% humidity, 80-90% of the solid compound I with amorphous form B is gradually transformed into stable crystalline form A. The compound of crystalline form A is a loose solid. Because the dosage of the compound I for adults with AIDS is only 1-3 mg per day, which is a low amount, the uniform mixing of the compound with an excipient is crucial to ensure the anti HIV effect of the drug. The compound I in the form of sheet amorphous solid B is not easy to mix evenly with excipients (see Table 1), which adversely affects the controllability of drug efficacy. The crystalline form A of the compound I is more stable than the amorphous crystalline form B, and is easy to mix evenly with excipients, which is conducive to drug quality control and drug efficacy.

TABLE 1
Content of compound I with amorphous B and crystalline form A in
tablets

| | | | | Number of tablets |
|---|---|---|---|---|
| | Crystalline | | Drug content for | arbitrarily |
| Compound | form | Static | each tablet* | selected |
| Crystalline form | Amorphous | Yes | 0.5-1.5 mg | 10 |
| of compound I | B | | | |
| | Crystalline | No | 0.95-1.05 mg | 10 |
| | form | | | |

*Each tablet contains an average of 1 mg of the compound I and 149 mg of excipients. The total weight of each tablet is 150 mg.

The results of single crystal study show that the crystalline form A of the compound I is monoclinic system and the space group is p2₁. The single crystal structure is as follows:

TABLE 2
Characterization data of single crystal A of compound I

| | |
|---|---|
| Identification of compound | Compound I |
| Empirical formula | C9 H11 F N6 O4 |
| Formula weight | 286.24 |
| Temperature | 303(2) K. |
| Wavelength | 1.54178 Å |
| Crystal system | Monoclinic |
| Space group | P21 |
| Unit cell dimensions | a = 11.0901(10) Å α = 90°. |
| | b = 9.3505(8) Å β = 109.608(4)°. |
| | c = 12.0032(10) Å γ = 90°. |
| Volume | 1172.53(18) Å3 |
| Z | 4 |
| Density (calculated) | 1.621 Mg/m³ |

Further studies showed the compound I had significant inhibitory effects on many types of cancer, and its anticancer activity and anti-lymphatic cancer effect were verified in animal models. Experiments showed that the compound I had significant inhibitory effects on B-cell non-Hodgkin's lymphoma, lung cancer and leukemia, especially on non-Hodgkin's lymphoma.

The following advantages are associated with the crystalline form A of 2′-fluoro-4′-substituted nucleoside analogue I of the disclosure. The 2′-fluoro-4′-substituted nucleoside analogues I have special structures including fluorine-containing groups and azide, which solves the shortcomings of toxic and side effects and low activity of conventional D- and L-nucleoside analogues, and when applied to the preparation of anti HBV or anti HCV or anti HIV drugs and anti-tumor drugs, especially drugs fighting against lymphatic cancer and non-Hodgkin's lymphatic cancer, and exhibit good application value. The crystalline form A of the compound has good stability, no static electricity, and is easy to mix evenly with excipients, which is conducive to the preparation of pharmaceutical preparations. The crystalline form A of the compound I is mixed with excipients to significantly improve its uniformity in the tablets, which is conducive to drug quality control, reducing the generation of drug resistance, and ensuring the efficacy. Therefore, the crystalline form A of the compound I of the disclosure can be used for preparing antiviral drugs, especially for preparing anti AIDS drugs. The crystalline form A of the compound I can also be used for preparing anti-cancer drugs, especially for preparing drugs for fighting against B-cell non-Hodgkin's lymphoma, lung cancer, leukemia and so on.

### BRIEF DESCRIPTION OF THE DRAWINGS

FIG. 1 is a differential scanning calorimetry (DSC)-thermogravimetric analysis (TGA) spectrum of a crystalline form A of a compound I of the disclosure;

FIG. 2 is a CuKα-X-ray powder diffraction (XRPD) spectrum of a crystalline form A of a compound I of the disclosure;

FIG. 3 is a stability comparison of CuKα-XRPD spectra of a crystalline form A of a compound I of the disclosure under high temperature: line 1 shows the CuKα-XRPD spectrum of the crystalline form A of the compound I; line 2 shows the CuKα-XRPD spectrum of the crystalline form A of the compound I at 60° C. for five days; and line 3 shows the CuKα-XRPD spectrum of the crystalline form A of the compound I at 60° C. for ten days;

FIG. 4 is a stability comparison of CuKα-XRPD spectra of a crystalline form A of a compound I of the disclosure under high humidity: line 1 shows the CuKα-XRPD spectrum of the crystalline form A of the compound I; line 2 shows the CuKα-XRPD spectrum of the crystalline form A of the compound I at 40° C. and 75% humidity for ten days; and line 3 shows the CuKα-XRPD spectrum of the crystalline form A of the compound I at 40° C. and 75% humidity for five days;

FIG. 5 is a stability comparison of CuKα-XRPD spectra of a crystalline form A of a compound I of the disclosure under light condition: line 1 shows the CuKα-XRPD spectrum of the crystalline form A of the compound I; line 2 shows the CuKα-XRPD spectrum of the crystalline form A of the compound I at 4500 lux for ten days; and line 3 shows the CuKα-XRPD spectrum of the crystalline form A of the compound I at 4500 lux for five days;

FIG. 6 is a stability comparison of CuKα-XRPD spectra of a crystalline form B of a compound I of the disclosure under high temperature: line 1 shows the CuKα-XRPD spectrum of the crystalline form B of the compound I; line 2 shows the CuKα-XRPD spectrum of the crystalline form B of the compound I at 60° C. for ten days; and line 3 shows the CuKα-XRPD spectrum of the crystalline form B of the compound I at 60° C. for five days;

FIG. 7 is a stability comparison of CuKα-XRPD spectra of a crystalline form B of a compound I of the disclosure under high humidity: line 1 shows the CuKα-XRPD spectrum of the crystalline form B of the compound I; line 2 shows the CuKα-XRPD spectrum of the crystalline form B of the compound I at 40° C. and 75% humidity for ten days; and line 3 shows the CuKα-XRPD spectrum of the crystalline form B of the compound I at 40° C. and 75% humidity for five days;

FIG. 8 is a stability comparison of CuKα-XRPD spectra of a crystalline form B of a compound I of the disclosure under light condition: line 1 shows the CuKα-XRPD spectrum of the crystalline form B of the compound I; line 2 shows the CuKα-XRPD spectrum of the crystalline form B of the compound I at 4500 lux for ten days; and line 3 shows the CuKα-XRPD spectrum of the crystalline form B of the compound I at 4500 lux for five days; and

FIG. 9 is a picture showing anti-T-cell lymphoma activity of the crystalline form A of the compound I of the disclosure in patient-derived tumor xenografts (PDX) mouse model.

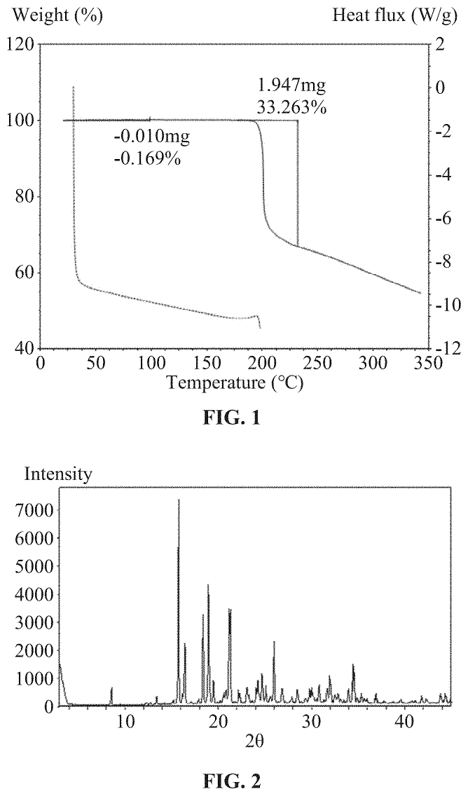

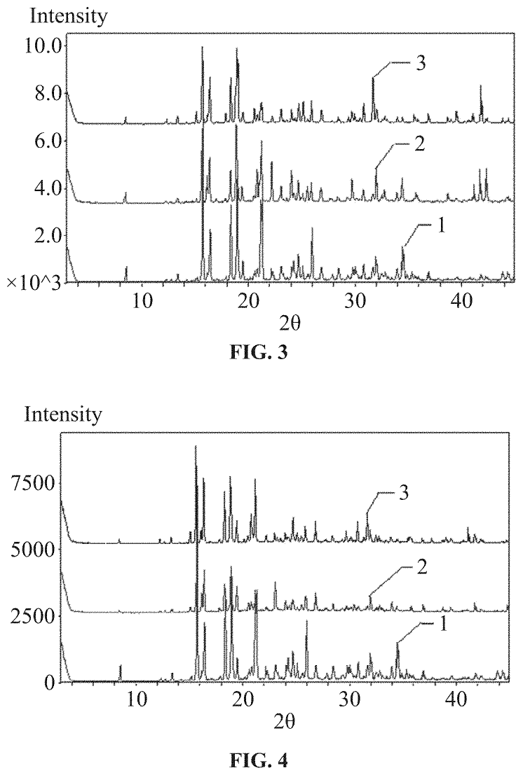

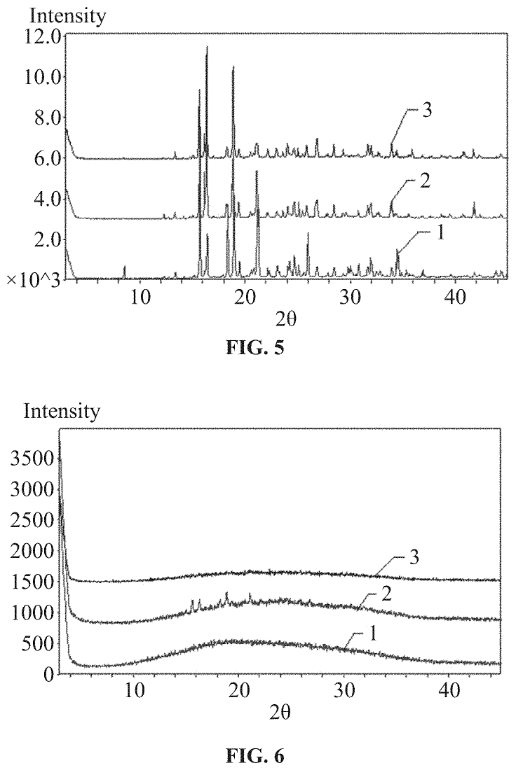

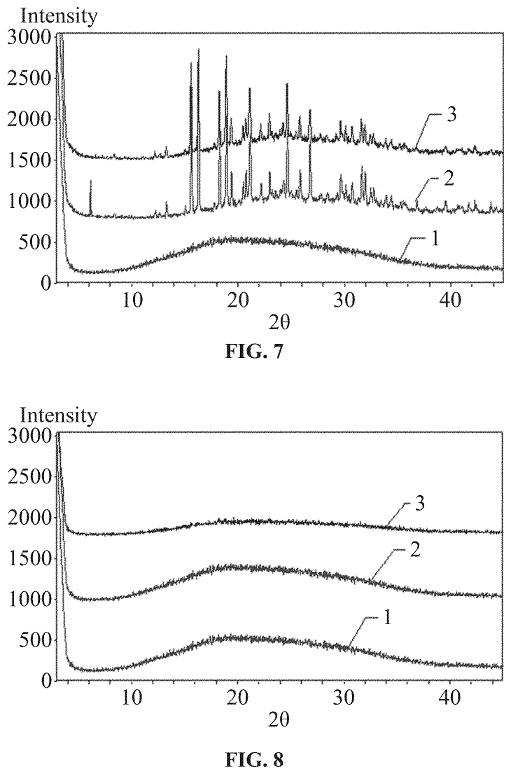

### DETAILED DESCRIPTION

To further illustrate, embodiments detailing a crystalline form A of 2′-fluoro-4′-substituted nucleoside analogue I are described below. It should be noted that the following embodiments are intended to describe and not to limit the disclosure.

### Example 1 Preparation of Crystalline Form B

1.1 Suspension Experiment

2′-fluoro-4′-substituted nucleoside analogue I of the disclosure was dissolved in one of the following solvents, and the solution concentration was 7-20 mg/mL. The solvent was removed using a rotatory evaporator, to yield a crystalline form B.

The abovementioned solvent is: methanol, ethanol, n-propanol, isopropanol, N,N-dimethylformamide (DMF), ethyl acetate, isopropyl acetate, n-hexane, cyclohexane, water, ether, isopropyl ether, methyl tert butyl ether, 4-methyl-pentanone, tetrahydrofuran, acetonitrile, dichloromethane and chloroform.

1.2 Rapid Evaporation Experiment

100 mg of the compound I was added to excess methanol solution and heated up for dissolution. The mixed solution was placed in a vacuum rotatory evaporator at 50° C. After the solvent was removed, a white solid was obtained, which was in the form of crystalline form B.

### Example 2 Preparation of Crystalline Form a

2.1 Recrystallization Experiment

2′-fluoro-4′-substituted nucleoside analogue I of the disclosure was dissolved in methanol, ethanol, n-propanol or water and heated to reflux to prepare its saturated solution. Thereafter, the saturated solution was cooled and crystallized to obtain the crystalline form A.

2.2 Liquid Level Diffusion Experiment

The compound I was dissolved in a solvent at 60° C., and then an antisolvent was slowly added dropwise to the sample solution along the wall, cooled, and a solid was precipitated. The crystalline form of the obtained solid is crystalline form A.

The solvent was Methanol, DMF or water.

The antisolvent was N-hexane, cyclohexane, isopropyl ether or ethyl acetate.

2.3 Binary Solvent Experiment

15 mg of the compound I was added to a sample bottle, and then 3 mL of a binary mixed solvent was added. The solution was shaken in a 200-rpm shaker at 50° C. for 48 h, and then filtered. The solution was allowed to slowly volatilize to remove the solvent, and the crystalline form A was obtained.

Binary mixed solvent: methanol (n-propanol, ethyl acetate, n-hexane, cyclohexane, isopropyl ether, methyl tert-butyl ether (MTBE), acetone, acetonitrile, dichloromethane or toluene), DMF (n-propanol, ethyl acetate, n-hexane, cyclohexane, isopropyl ether, MTBE, acetone, acetonitrile, dichloromethane or toluene), water (n-propanol, ethyl acetate, n-hexane, cyclohexane, isopropyl ether, MTBE, acetone, acetonitrile, dichloromethane or toluene); the ratio of the binary mixed solvent:methanol:n-propanol=1:4 (volume ratio), DMF:n-propanol=1:4 (volume ratio), water:n-propanol=1:4 (volume ratio), and so on.

The experimental results showed that the stable crystalline form of the compound I was crystalline form A. The crystalline form A of the compound I had diffraction peaks at 20 angles (±0.2): 8.56, 13.40, 15.76, 16.43, 18.38, 18.95, 19.49, 20.62, 20.86, 21.20, 25.99, 26.85, 27.89, 28.48, 29.78, 30.04, 30.84, 31.71, 31.96, 33.95, 34.47 through X-ray powder diffraction (XRPD) using CuKα radiation with a wavelength of λ=1.5418 Å (as shown in FIG. 2).

Diffraction peaks at 2θ angles and corresponding relative intensities (%) of the crystalline form A of the compound I:

| 2θ (±0.2°) | Relative intensities I % |
|---|---|
| 8.56 | 8.6 |
| 13.40 | 4.2 |
| 15.76 | 100 |
| 16.43 | 29.5 |
| 18.38 | 42.7 |
| 18.95 | 58.1 |
| 19.49 | 10.7 |
| 20.62 | 4.9 |
| 20.86 | 6.2 |
| 21.20 | 45.8 |
| 25.99 | 29.4 |
| 26.85 | 7.0 |
| 27.89 | 2.5 |
| 28.48 | 6.2 |
| 29.78 | 6.0 |
| 30.04 | 6.8 |
| 30.84 | 8.0 |
| 31.71 | 6.7 |
| 31.96 | 12.9 |
| 33.95 | 6.2 |
| 34.47 | 18.7 |

### Conclusion

1. The results of DSC and XRD showed that the crystalline form A was the stable crystalline form of the compound I.

2. The crystalline form A exhibited good stability in stability evaluation.

3. The crystalline form B was generally stable and can be transformed into crystalline form A under appropriate conditions.

4. The crystalline form B was transformed into crystalline form A under high humidity and high temperature conditions for ten days.

5. When the crystalline form A of the compound I was mixed with excipients, its uniformity in the tablets was significantly improved, which is conducive to the quality control of drugs, reducing the generation of drug resistance, and ensuring the efficacy.

### Example 3 Antiviral Effect of Crystalline Form A of Compound I

The anti HIV activity of crystalline form A of the compound I was determined according to the literature method (EUR. J. Med. Chem. 2011, 464178).

### Example 4 Inhibitory Effect of Crystalline Form A of Compound I

The anticancer (non-Hodgkin's lymphoma) activity of the crystalline form A of the compound I was determined according to the literature method (Asian Pacific Journal of cancer prevention 2014, 15, 6829).

### Example 5 Anticancer Effect of the Crystalline Form a of the Compound I on B-Cell Non-Hodgkin Lymphoma, Lung Cancer, Gastric Cancer, Colorectal Cancer and Leukemia

The anticancer activity of the crystalline form A of the compound I on B-cell non-Hodgkin lymphoma, lung cancer, gastric cancer, colorectal cancer and leukemia was determined according to the literature method (Wang, Q. et al Biochemical Pharmacology 2011, 81, 848; Asian Pacific Journal of Cancer Prevention 2014, 15, 6829).

### Example 6 Anti-T-Cell Lymphoma Activity of the Crystalline Form A of the Compound I in Patient-Derived Tumor Xenografts (PDX) Mouse Model

The inhibitory effect of the crystalline form A of the compound I on T-cell lymphoma was determined by patient-derived tumor xenograft (PDX).

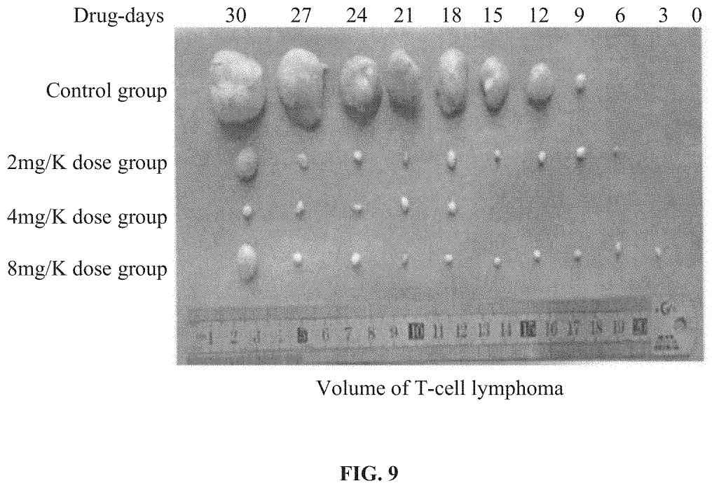

After the patient's T-cell lymphoid carcinoma tissue was sampled, the tissue was transferred to a specific pathogen free (SPF) animal room through a transfer window. In a super clean table, phosphate buffered saline (PBS) buffer with double antibodies, Dulbecco's modified eagle medium (DMEM) medium, Petri dish and ice bag were prepared. An appropriate amount of PBS and DMEM were poured to the Petri dishes on ice. The tissue was taken out and put into PBS for preliminary cleaning, and necrotic and normal tissue was removed. The tissue in PBS was transferred to DMEM. The remaining tissue was cut into small pieces of 20-30 mm³ patches, which were transplanted into the armpit and back of mice with puncture needles. Each mouse was inoculated with the tissue on one site. The tumors of the inoculated mice were taken out regularly, photographed, and the volume changes thereof were compared (see FIG. 9).

Through the experiments of patient-derived tumor xenografts mouse model, the crystalline form A of the compound I could significantly inhibit the growth of T-cell lymphoma at the doses of 2 mg/kg, 4 mg/kg, and 8 mg/kg, respectively.

It will be obvious to those skilled in the art that changes and modifications may be made, and therefore, the aim in the appended claims is to cover all such changes and modifications.

What is claimed is:

1. A crystalline form A of a compound I, which is a monoclinic system and a space group thereof is p2₁, and a single crystal structure being as follows:

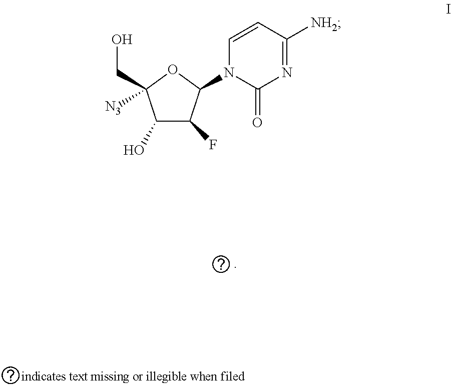 [SMILES: [N-]=[N+]=N[C@]1(CO)O[C@@H](n2ccc(N)nc2=O)[C@@H](F)[C@@H]1O]

2. The crystalline form of claim 1, having diffraction peaks in a CuKα-X-ray powder diffraction (XRPD) spectrum when a diffraction angle 20 is: 8.56, 13.40, 15.76, 16.43, 18.38, 18.95, 19.49, 20.62, 20.86, 21.20, 25.99, 26.85, 27.89, 28.48, 29.78, 30.04, 30.84, 31.71, 31.96, 33.95, 34.47; and an error range of 20 is ±0.2.

3. The crystalline form of claim 1, wherein the CuKα-X-ray powder diffraction (XRPD) spectrum is shown in FIG. 2.

4. The crystalline form of claim 2, wherein the CuKα-X-ray powder diffraction (XRPD) spectrum is shown in FIG. 2.

5. A method for preparation of an antiviral drug comprising applying the crystalline form A of the compound I of claim 1.

6. The method of claim 5, wherein the antiviral drug is an anti-HIV drug.

7. A method for preparation of an antitumor drug comprising applying the crystalline form A of the compound I of claim 1.

8. The method of claim 7, wherein the antitumor drug is used as an active ingredient for treatment of lung cancer, gastric cancer, colorectal cancer, leukemia, or lymphatic cancer.

9. The method of claim 8, wherein the antitumor drug is used as an active ingredient for treatment of B-cell non-Hodgkin's lymphoma.
